(19) United States
(12) Patent Application Publication
(10) Pub. No.: US 2025/0295559 A1
(43) Pub. Date: Sep. 25, 2025

(54) MEDICAL MIXER SYSTEM

(71) Applicant: Staysis Medical, LLC, Scottsdale, AZ (US)

(72) Inventors: Rui J. Ferreira, Livingston, NJ (US); Frank J. Schwab, Oldwick, NJ (US); Shay Bess, Parker, CO (US)

(73) Assignee: Staysis Medical, LLC, Scottsdale, AZ (US)

(21) Appl. No.: 19/081,220

(22) Filed: Mar. 17, 2025

Related U.S. Application Data
(60) Provisional application No. 63/567,146, filed on Mar. 19, 2024.

(51) Int. Cl. A61J 1/20

(52) U.S. Cl. CPC A61J 1/2096; A61J 1/2058; A61J 1/2065

(57) ABSTRACT

A medical mixer apparatus having a vial holder, a cap, and an adapter defining a first passageway and second passageway. In a first position of the adapter, a connector is aligned with the first passageway and a chamber in the vial holder. In a second position of the adapter, the connector, second passageway, and another connector are aligned.

### CROSS-REFERENCE TO RELATED APPLICATIONS

This application claims the benefit of U.S. Provisional Application Ser. No. 63/567,146, filed Mar. 19, 2024, the disclosure of which is hereby incorporated by reference herein in its entirety.

### TECHNICAL FIELD

The invention generally relates to systems and methods for mixing medical products that are used in surgery, or in other medical procedures. The medical products can include hemostasis agents, medications, bone growth materials, and other materials.

### BACKGROUND

In providing materials for medical procedures using existing technologies, some materials are provided as a powder, gel, liquid or some other form, and mixing with saline, gelatin, or other liquid or non-liquid materials may be required. For example, a hemostat like thrombin or tranexamic acid (TXA) is provided in a powdered form because once reconstituted with saline and gelatin, it must be used in 3 hours. This requires the operating room staff to mix the thrombin into a paste that can be delivered to a surgical site. The thrombin, or other medical product or pharmaceutical, is provided in a vial or other container that can be sterile or non-sterile. The vial, or container, is carefully pierced or gained access to, and the thrombin (or other powdered medical product) is mixed with saline, which is provided in a vial or syringe. The reconstituted thrombin is disposed in a syringe and attached to another syringe containing gelatin and the material is driven back and forth between the two syringes to mix the gelatin and thrombin into a paste that can be delivered to a surgical site. The consistency of the mixed paste varies. If the thrombin is provided non-sterile, it can be challenging to reconstitute the thrombin and then deliver it into the sterile field. Similar challenges exist in mixing other medical products that may need to be reconstituted, mixed, or combined with other materials and formed into a paste or liquid, or provided in another form.

### SUMMARY

In an aspect, a medical mixer apparatus comprises a vial holder having a chamber, a first connector, and an adapter defining a first passageway, the adapter being movable to a position in which the first passageway extends from the first connector to the chamber.

In embodiments, the apparatus can have a vial wherein the vial can be disposed in the vial holder. The apparatus can have a cap attachable to a first end of the vial holder. In embodiments, the vial holder has a second end opposite the first end and a spike at the second end.

In embodiments, the adapter has a second passageway and the apparatus further comprises a second connector.

In embodiments, the adapter is movable from a first position in which the chamber and the first connector are aligned with the first passageway to a second position in which the first connector and second connector are aligned with the second passageway.

In embodiments, at least one of the first connector and second connector is arranged to connect to a syringe. The first connector can be arranged to connect to a first syringe and the second connector can be arranged to connect to a second syringe. In embodiments, the syringe contains saline.

In embodiments, the second passageway has at least one bump defined therein. In embodiments, the second passageway has at least one curved ridge defined therein.

In embodiments, the adapter is rotatable.

In embodiments, the second passageway is disposed beneath the first passageway.

In embodiments, the adapter is rotatable and the adapter moves up with respect to the vial holder when rotated to align the first connector and second connector with the second passageway.

In a further aspect, a kit comprises saline in a first syringe, another substance in a vial, and a medical mixer apparatus. The medical mixer apparatus comprises a vial holder having a chamber, a first connector, and an adapter defining a first passageway, the adapter being movable to a position in which the first passageway extends from the first connector to the chamber.

In an aspect, a method of mixing a medical product comprises providing the kit, and mixing the saline and the other substance using the medical mixer apparatus. The kit comprises saline in a first syringe, another substance in a vial, and a medical mixer apparatus. In embodiments, the method further comprises rotating the adapter to move the adapter.

In an aspect, a kit comprises saline in a first syringe, another substance in a vial, a second syringe, and a medical mixer apparatus. The medical mixer apparatus comprises a vial holder having a chamber, a first connector, and an adapter defining a first passageway, the adapter being movable to a position in which the first passageway extends from the first connector to the chamber. The adapter can have a second passageway and a second connector. The adapter can be movable from a first position in which the chamber and the first connector are aligned with the first passageway to a second position in which the first connector and second connector are aligned with the second passageway.

In an aspect, a method of mixing a medical product comprises providing the kit and mixing the saline and the other substance using the medical mixer apparatus. The kit comprises saline in a first syringe, another substance in a vial, a second syringe, and a medical mixer apparatus. The medical mixer apparatus comprises a vial holder having a chamber, a first connector, and an adapter defining a first passageway, the adapter being movable to a position in which the first passageway extends from the first connector to the chamber. The adapter can have a second passageway and a second connector. The adapter can be movable from a first position in which the chamber and the first connector are aligned with the first passageway to a second position in which the first connector and second connector are aligned with the second passageway.

In embodiments, the method includes rotating the adapter to move the adapter from the first position to the second position.

In an aspect, a medical mixer apparatus comprises a vial holder having a first end, a second end, and a chamber between the first end and the second end, a cap attachable to the first end, and an adapter movable from a first position to a second position. The adapter defines a first passageway, a second passageway, a first connector, and a second connector. The first passageway extends from the second connector to the chamber when the adapter is in the first position, and the second passageway extends from the first connector to the second connector when the adapter is in the second position.

In embodiments, at least one of the first connector and second connector is arranged to connect to a syringe. The first connector can be arranged to connect to a first syringe and the second connector can be arranged to connect to a second syringe.

In embodiments, a spike is disposed in the chamber at the second end. The cap may have an end for abutting a surface of a vial. In an embodiment, the cap and vial holder have attachment features arranged so that a vial is pierced by the spike when the cap is attached and a vial is disposed in the chamber.

In embodiments, the second passageway has at least one bump defined therein. The second passageway can have at least one curved ridge defined therein.

In embodiments, the adapter is rotatable. In embodiments, the adapter is rotatable to move from the first position to the second position. In an example, the second passageway is disposed beneath the first passageway. For example, the adapter can move up with respect to the vial holder when rotated to align the first connector and second connector with the second passageway.

In further aspects, a kit is provided. The kit can comprise saline in a first vial, a medical product in a second vial, another substance in a first syringe, a second syringe, and a medical mixer apparatus. The medical mixer apparatus can comprise a vial holder having a first end, a second end, and a chamber between the first end and the second end, a cap attachable to the first end, and an adapter movable from a first position to a second position. The adapter can define a first passageway, a second passageway, a first connector, and a second connector. For example, the first passageway extends from the second connector to the chamber when the adapter is in the first position, and the second passageway extends from the first connector to the second connector when the adapter is in the second position.

In another aspect, a kit comprises a medical product in a vial, another substance in a first syringe, a second syringe containing saline, and a medical mixer apparatus. The medical mixer apparatus can comprise a vial holder having a first end, a second end, and a chamber between the first end and the second end, a cap attachable to the first end, and an adapter movable from a first position to a second position. The adapter can define a first passageway, a second passageway, a first connector, and a second connector. For example, the first passageway extends from the second connector to the chamber when the adapter is in the first position, and the second passageway extends from the first connector to the second connector when the adapter is in the second position.

In a further aspect, a kit comprises saline in a first syringe, another substance in a vial, a second syringe, and a medical mixer apparatus. The medical mixer apparatus can comprise a vial holder having a first end, a second end, and a chamber between the first end and the second end, a cap attachable to the first end, and an adapter movable from a first position to a second position. The adapter can define a first passageway, a second passageway, a first connector, and a second connector. For example, the first passageway extends from the second connector to the chamber when the adapter is in the first position, and the second passageway extends from the first connector to the second connector when the adapter is in the second position.

A further aspect is a method of mixing a medical product, comprising providing a kit and mixing the medical product and the other substance using the medical mixer apparatus. The kit can comprise saline in a first vial, a medical product in a second vial, another substance in a first syringe, a second syringe, and a medical mixer apparatus. The medical mixer apparatus can comprise a vial holder having a first end, a second end, and a chamber between the first end and the second end, a cap attachable to the first end, and an adapter movable from a first position to a second position. The adapter can define a first passageway, a second passageway, a first connector, and a second connector. For example, the first passageway extends from the second connector to the chamber when the adapter is in the first position, and the second passageway extends from the first connector to the second connector when the adapter is in the second position.

In embodiments, the adapter rotated to move the adapter from the first position to the second position.

In another aspect, a kit comprises a substance in a first syringe, a second syringe containing saline, and a medical mixer apparatus. The medical mixer apparatus can comprise a vial holder having a first end, a second end, and a chamber between the first end and the second end, a cap attachable to the first end, and an adapter movable from a first position to a second position. The adapter can define a first passageway, a second passageway, a first connector, and a second connector. For example, the first passageway extends from the second connector to the chamber when the adapter is in the first position, and the second passageway extends from the first connector to the second connector when the adapter is in the second position.

In another aspect, a method of mixing a medical product comprises providing a kit and mixing the saline and the other substance using the medical mixer apparatus. The kit comprises saline in a vial, another substance in a first syringe, a second syringe, and a medical mixer apparatus. The medical mixer apparatus can comprise a vial holder having a first end, a second end, and a chamber between the first end and the second end, a cap attachable to the first end, and an adapter movable from a first position to a second position. The adapter can define a first passageway, a second passageway, a first connector, and a second connector. For example, the first passageway extends from the second connector to the chamber when the adapter is in the first position, and the second passageway extends from the first connector to the second connector when the adapter is in the second position.

In embodiments, the method further comprises rotating the adapter to move the adapter from the first position to the second position. The method can include attaching a first syringe to the first connector, attaching a second syringe to the second connector, moving the adaptor to the second position, and passing a material from the first syringe to the second syringe at least once.

The above-described benefits, embodiments, and or characterizations are not necessarily complete or exhaustive. Other benefits, embodiments, and or characterizations of the present disclosure are possible.

### BRIEF DESCRIPTION OF THE DRAWINGS

The accompanying drawings, which are incorporated in and constitute a part of the present disclosure, illustrate preferred embodiments of the invention and, together with the description, serve to explain the present disclosure. Embodiments of the invention are in no way limited by the following figures:

FIG. 1 is a front perspective view of the medical mixer apparatus according to certain embodiments;

FIG. 2 is a cross-sectional view of the medical mixer apparatus of FIG. 1;

FIG. 3 is a front elevation view of the medical mixer apparatus of FIGS. 1 and 2;

FIG. 4A is a cross-sectional view of the medical mixer apparatus according to further embodiments;

FIG. 4B is a detail of FIG. 4A;

FIG. 5 is a front perspective view of a medical mixer apparatus according to another embodiment;

FIG. 6 is a detail of a medical mixer apparatus according to further embodiments;

FIG. 7A is a bottom view of a pedicle tip in accordance with certain embodiments;

FIG. 7B is a left elevation view of the pedicle tip of FIG. 7A;

FIG. 7C is a front plan view of the pedicle tip of FIGS. 7A-7B;

FIG. 7D is a front perspective view of the pedicle tip of FIGS. 7A-7C;

FIG. 8A is a bottom view of a pedicle tip in accordance with other embodiments;

FIG. 8B is a left elevation view of the pedicle tip of FIG. 8A;

FIG. 8C is a front plan view of the pedicle tip of FIGS. 8A-8B;

FIG. 8D is a front perspective view of the pedicle tip of FIGS. 8A-8C;

FIG. 9A is a bottom view of a pedicle tip in accordance with other embodiments;

FIG. 9B is a left elevation view of the pedicle tip of FIG. 9A;

FIG. 9C is a front plan view of the pedicle tip of FIGS. 9A-9B;

FIG. 9D is a front perspective view of the pedicle tip of FIGS. 9A-9C;

FIG. 10 is a front perspective view of the pedicle tip of FIGS. 9A-9D; and

FIG. 11 is a front perspective view of a pedicle tip according to further embodiments.

### DETAILED DESCRIPTION

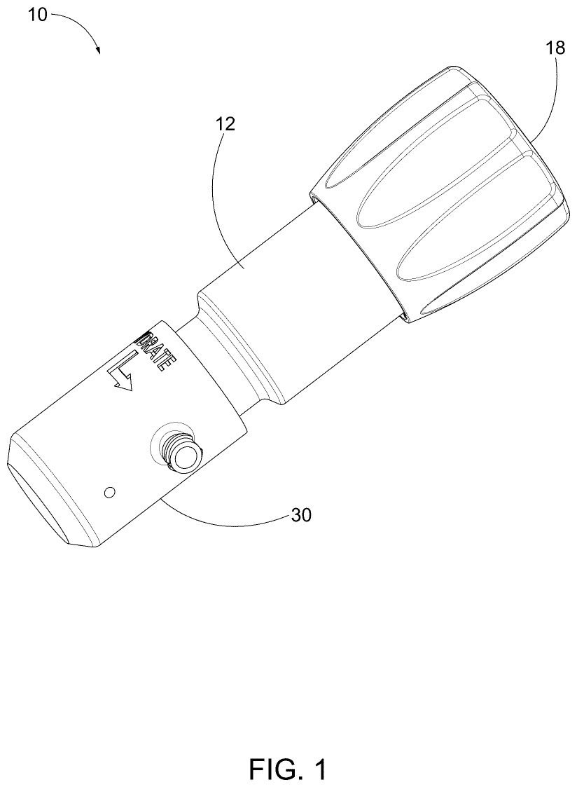

A medical mixer apparatus 10 according to certain embodiments is shown in FIG. 1. The apparatus 10 has a vial holder 12 with a chamber 14 for holding a medical product provided in a vial 16, which may be sterile or non-sterile. The apparatus itself is sterile, protecting the sterile field from a non-sterile vial. For example, the vial 16 may contain thrombin in a powdered form, but any other medical product that has to be mixed is also contemplated. The product, products, component, etc., can be liquid, gel, powder, etc.

In any of the embodiments disclosed herein, the medical product can be thrombin, TXA, bone matrix, demineralized bone matrix (DBM), platelet rich plasma (PRP), allograft, autograft, peptides, bone morphogenic protein, artificial bone or bone growth products, antibiotics, pharmaceuticals, synthetic graft materials, agents to control bleeding, bone marrow, blood, putty, and other biologic or synthetic products. It is contemplated that a medical mixing apparatus in accordance with embodiments hereof can be used to mix antibiotics or growth factors with any combination of gelatin, collagen, DBM, PRP, etc..

The apparatus 10 has a cap 18 that can be attached to the vial holder 12 by threaded connection, snap-on connection, bayonet connection, or other means. The cap 18 has an end 20 that is arranged to abut a bottom surface 22 of the vial 16. The chamber 14 of the vial holder 12 has a bottom end 24 that receives the vial. In embodiments, the bottom end has a pointed spike 26 or other sharp feature for piercing the vial (the vial may have a septum 28). In embodiments, attaching the cap 18 to the vial holder 12 drives the end 20 against the vial bottom 22, driving the septum 28 into the pointed spike 26 and piercing the vial 16. In other words, the distance between the end 20 when the cap 18 is fully seated on the vial holder 12 and the bottom end 24 is such that the spike 26 will pierce the septum 28. The pointed spike 26 may be non-coring in design, meaning that its design may be such that it will not remove a piece of the septum when it pierces it. The chamber 14 of the vial holder 12 and the operation of the attachment of the cap 18, in embodiments, drive the pointed tip 26 or other sharp feature into the septum 28 in a linear fashion, with no relative angulation therebetween.

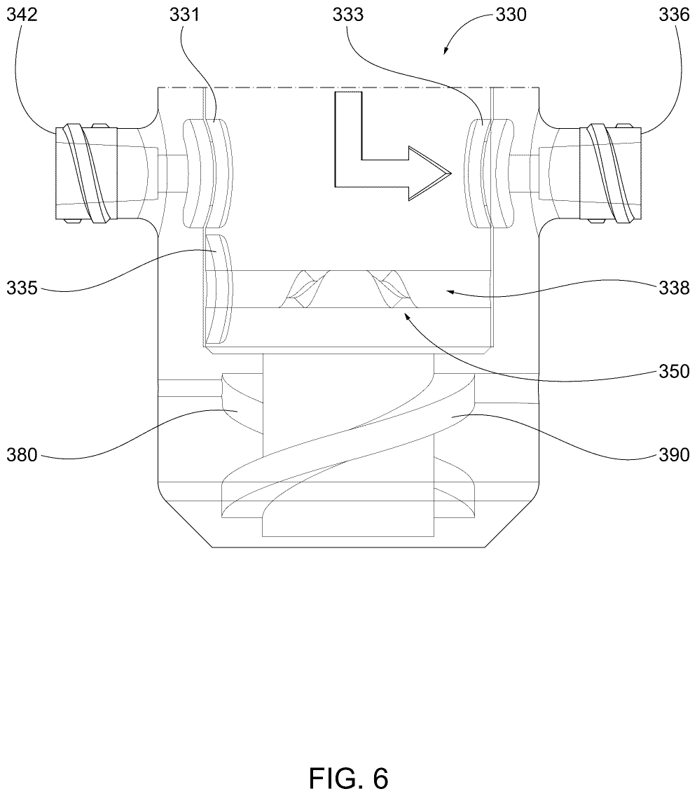

The apparatus 10 has an adapter 30 that is movably attached to a bottom end 32 of the vial holder 12. In embodiments, the adapter 30 defines two passageways and two connectors. The first passageway 34 extends from a first connector 36, which may be a Luer connector, to the chamber 14 and punctured vial 16. The second passageway 38 extends from a first connector 36 to the second connector 42. The first connector 36 may be a Luer connector. In embodiments, the second passageway 38 is disposed below the first passageway 34 in the adapter 30. In embodiments, the first and second connectors are of a type suitable for having a syringe attached thereto. The adapter 30 is movable to connect the first connector 36 to the first passageway 34 in a first position, and movable to connect the first and second connectors to the second passageway 38 in a second position. In embodiments, the adapter 30 is rotatable so that, by operation of threads, ramps, or other features (see FIG. 6), the adapter 30 moves while rotating to change which of the passageways are being used. In embodiments, the adapter 30 moves vertically to vary the alignment of the passageways. In this way, material can be delivered from the first connector 36 to the pierced vial 16, or material can be delivered from the first connector 36 to the second connector 42 and, if so desired, from the first connector 36 to the second connector 42. In embodiments, the adapter has more than two passageways.

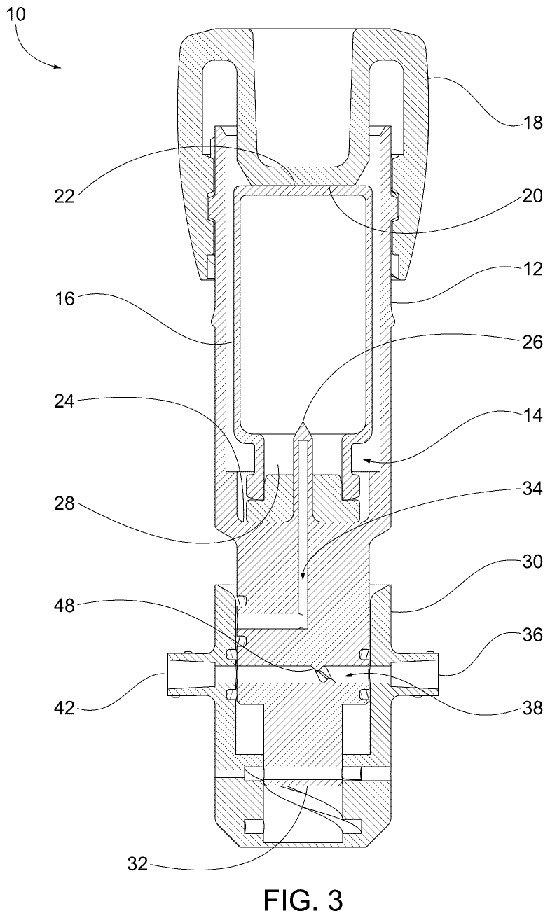

In an example of reconstituting powdered thrombin and mixing the thrombin into a paste, although the present description is not limited to mixing thrombin paste products and can be used to mix other medical products, a thrombin vial is disposed in the chamber 14 of the vial holder 12 and when the cap 18 is attached, the thrombin vial is pierced by the pointed spike 26. With the adapter 30 in the first position, the first connector 36 is connected to the first passageway 34 and communicates with the punctured vial. A first syringe 44 containing saline is attached to the first connector 36. Using the plunger on the syringe 44, saline is delivered to the thrombin and then reconstituted thrombin is drawn into the first syringe 44. Adjusting the adapter 30 moves the adapter so that the second passageway 38 is aligned with the first and second connectors. A second syringe 46 containing gelatin is attached to the second connector 42 (see FIG. 3). By operating the plungers on the syringes, the gelatin can be mixed with the reconstituted thrombin. It may be necessary to pass material from the first syringe 44 to the second syringe 46 and then from the second syringe 46 to the first syringe 44 several times. After being mixed in this fashion, a thrombin paste has been formed which is suitable for use in a surgical or other procedure. The mixed product can be delivered to a pedicle tip, nozzle, etc., using the syringe.

Because the vial 16 is disposed in the chamber 14 of the vial holder 12, and the medical mixer apparatus 10 is provided sterile, the vial 16 can be provided non-sterile and used in the sterile field, without compromising the sterile field.

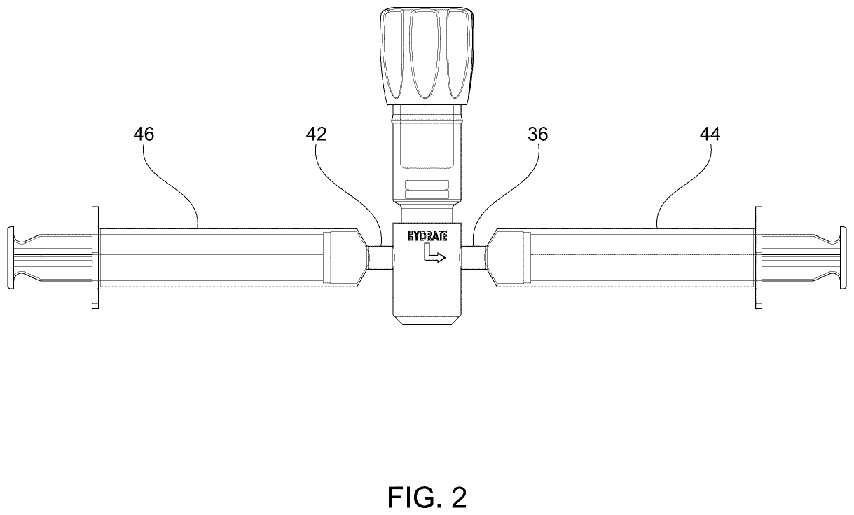

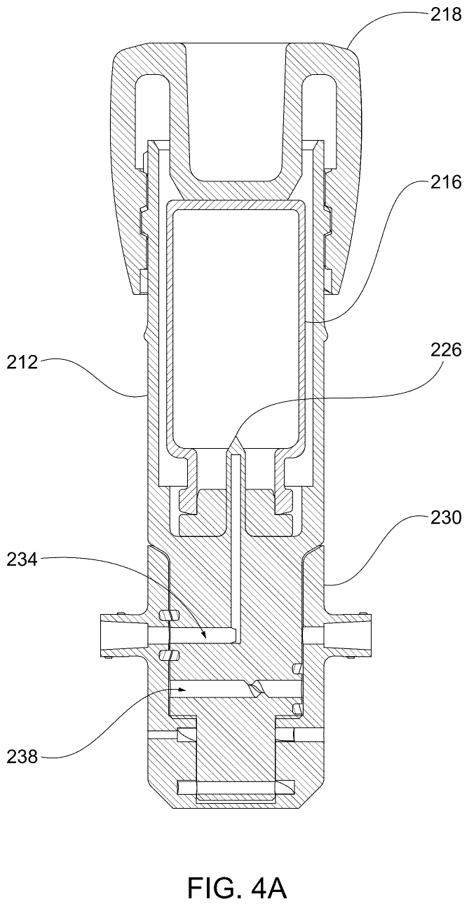

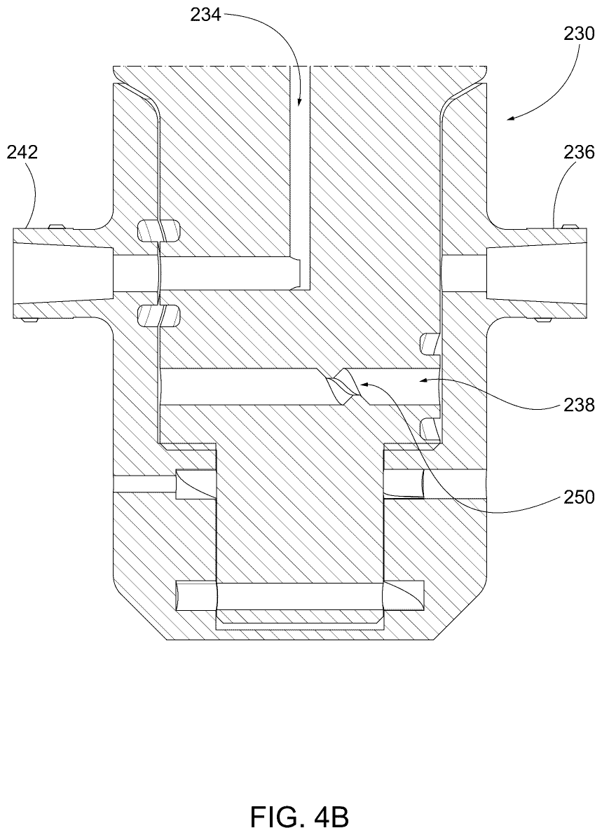

The second passageway 38, in embodiments, has features for promoting the mixing of medical substances. As shown in FIG. 2, at least one bump 48 is formed in the second passageway 38. In certain embodiments, two bumps are provided. Other obstructions having other shapes are contemplated. These features create some turbulence in the medical substances as they are passed through the second passageway 38. In another embodiment, at least one curved ridge is provided in the second passageway. In certain embodiments, two curved ridges 250 are provided on opposing sides of the second passageway. (See FIG. 4B). In certain embodiments, the curved ridges are helical or semi-helical in shape. In embodiments, one or more of the passageways have features for promoting the mixture of the medical product, or components thereof, such as bumps, ridges, curves, etc.

In further embodiments, one or both of the first and second syringes can be replaced with different containers. For example, the gelatin can be provided in a flexible pouch that delivers the gelatin when compressed by hand. In other embodiments, the reconstituted medical product can be disposed in a flexible pouch that is compressed by hand. These pouches can be attached to the medical mixer apparatus and used to mix a paste of the medical product. Other types of containers are contemplated.

In embodiments, the medication active agent is disposed in a vial and is mixed with saline from a syringe. The mixed product is transferred to another syringe to be administered. It is also possible that a syringe has some other component that is required to be introduced to the medication in the vial to make the medication active.

In embodiments, a kit comprises a medical product like thrombin in a vial, a syringe of saline, another syringe, and a mixer apparatus. In embodiments, one or more nozzles are provided based on the procedure to be performed.

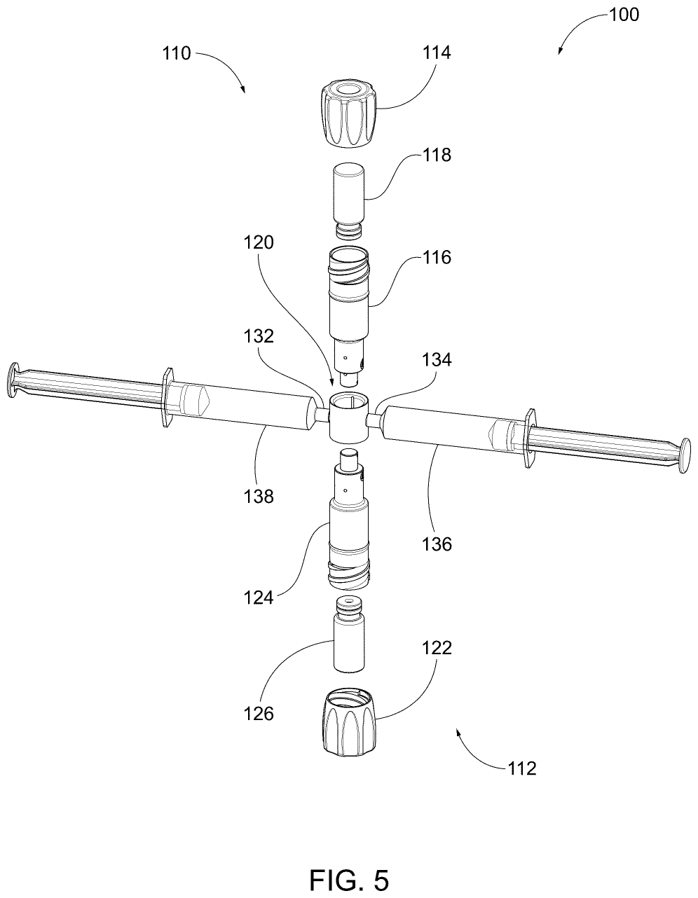

In further embodiments, shown for example in FIG. 5, a medical mixer apparatus 100 has a first side 110 and a second side 112. In embodiments, the first side 110 has a first cap 114, a first vial holder 116 for a first vial 118, and an adapter 120. The apparatus 100 has a second side 112 with a second cap 122, and a second vial holder 124 for a second vial 126, on the other side of the adapter 120. In embodiments, one or more of the vial holders have a pointed spike or other feature for opening a vial. The apparatus 100 has a first connector 132 and a second connector 134 for a first syringe 136 and a second syringe 138. The first vial 118 and second vial 126 can contain medical products such as pharmaceuticals, substances like gelatin, saline, thrombin, antibiotics, and the like, and these products and substances can be mixed by being passed back and forth between the first syringe 136 and a second syringe 138, or being passed between other types of containers attached at the first connector 132 and second connector 134. In certain embodiments, the adapter 120 defines a first passageway, a second passageway, and a third passageway. The first passageway can be on the first side for connecting the first vial holder with a connector, the second passageway is between the first passageway and second passageway for connecting the two connectors, and the third passageway can be on the second side for connecting the second vial holder to a connector. In this way, more than one medical product can be mixed together. In further embodiments, a medical mixer apparatus has more than two sides for mixing more than two medical products. In an example, thrombin is provided in a vial, antibiotics are provided in a second vial and medical mixer apparatus 100 is used. In another example, tranexamic acid is provided in the second vial.

In certain embodiments, a kit includes saline in a vial, a medical product in a vial, another substance in a first syringe, a second syringe, and a medical mixer apparatus. The second syringe can be used to draw the saline and introduce the saline through the medical mixer apparatus to the medical product, while the medical mixer apparatus is in a first position. The medical mixer apparatus can be moved to a second position, the second syringe can be attached to the medical mixer apparatus and the medical product can be mixed with the substance in the first syringe. In certain embodiments, a kit is as described above, except that the saline is provided in the second syringe. In certain embodiments, a kit includes saline in a vial, another substance in a first syringe, a second syringe, and a medical mixer apparatus. A medical product is obtained separately and mixed using the medical mixer apparatus. In certain embodiments, a kit includes saline in a syringe, another substance in a different syringe, and a medical mixer apparatus. A medical product is obtained separately and mixed using the medical mixer apparatus. In embodiments, containers other than syringes are used; kits may include one or more syringes, one or more containers, etc. In embodiments, a kit includes one or more pedicle tips, nozzles, etc.

FIGS. 4A and 4B show a medical mixer apparatus according to further embodiments. The apparatus is as discussed above in connection with FIGS. 1-3 having a cap 218, vial holder 212 for holding a vial 216, and an adapter 230. The vial holder has a pointed spike 221 for piercing the vial 216, a first connector 236, and second connector 242. The adapter 230 defines a first passageway 234 and second passageway 238 and is movable between a position in which the first passageway 234 is connected to the first connector 236 and to the chamber of the vial holder 212 that holds the vial 216 to a position in which the second passageway 238 is connected to the first connector 236 and second connector 242.

As shown in FIG. 4B, the second passageway 238 has at least one curved ridge 250. The curved ridge 250 can be helical or define part of a helix. In certain embodiments, the second passageway 238 has two ridges. In certain embodiments, the second passageway 238 has a first semi-helical ridge on a first side and a second semi-helical ridge on a second side. The ridges serve to provide turbulence to the liquid passing through the second passageway 238 during mixing of the medical product. The ridges have many advantages, including producing a mixed medical product with more consistent viscosity.

In all of the embodiments disclosed herein, the various parts of the medical mixer apparatus can be, optionally, formed with polymeric materials that are used for medical products, using stereolithographic, 3-D printing, injection molding, and other molding techniques.

It is contemplated that the medical mixer apparatus 10, according to its various embodiments, has a movable adapter that is slidable vertically, to move one or more passageways (such as, for example, the first passageway and/or second passageway) into position. In certain embodiments, the adapter and vial holder have features that provide a click-type sensory feedback alerting the user as to the position of the adapter in a correct operative position, fixing the position of the adapter, or both. For example, a detent engagement between the adapter and vial holder can be used. This feature can provide feedback that is felt and/or heard by the user.

In embodiments, a medical mixer apparatus has one or more vial holders and one or more passageways. Connectors for syringes or other types of containers can be included

In any of the embodiments disclosed herein, the medical mixer apparatus 10 can have an adapter that defines ramps, cams or threads arranged so that, upon rotation of the adapter, the adapter moves up (or down) to align the first passageway and second passageway with the first and/or second connectors. In embodiments, the adapter moves laterally to align one or more passageways. As shown in FIG. 6, for example, adapter 330 has a first connector 342 and a second connector 336, as well as a first passageway (not shown) and a second passageway 338. In certain embodiments, the second passageway 338 has a first semi-helical ridge 350 and a second semi-helical ridge. In certain embodiments, one or more seals are provided between the adapter 330 and vial holder. For example, the medical mixer apparatus has a first seal 331, second seal 333, and third seal 335 to prevent leakage of liquid between the adapter and vial holder. The seal may be an O-ring, gasket, or other means known to those skilled in the art.

As shown in FIG. 6, the adapter 330 defines one or more ramps, threads, or other angular features (such as first ramp 380 and second ramp 390) so that as the adapter is rotated, the adapter moves up, changing the position of the first passageway and second passageway 338 with respect to the first connector 342 and second connector 336. In certain embodiments, the vial holder defines posts that travel in the first ramp 380 and second ramp 390.

In certain embodiments, the connectors can be female Luer connectors, male Luer connectors, or any mix of them. In FIG. 6, the first connector 342 and second connector 336 are both female Luer connectors. It is contemplated that a kit, in certain embodiments, includes a medical mixer apparatus 10 with an adapter having one male Luer connector and a female Luer connector, in addition to a male syringe for the saline and a female syringe for the gelatin. In this way, the syringes can only be connected to the correct connector, avoiding confusion and possible error.

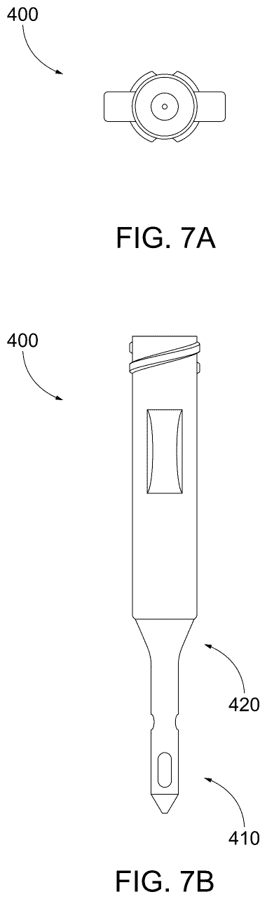

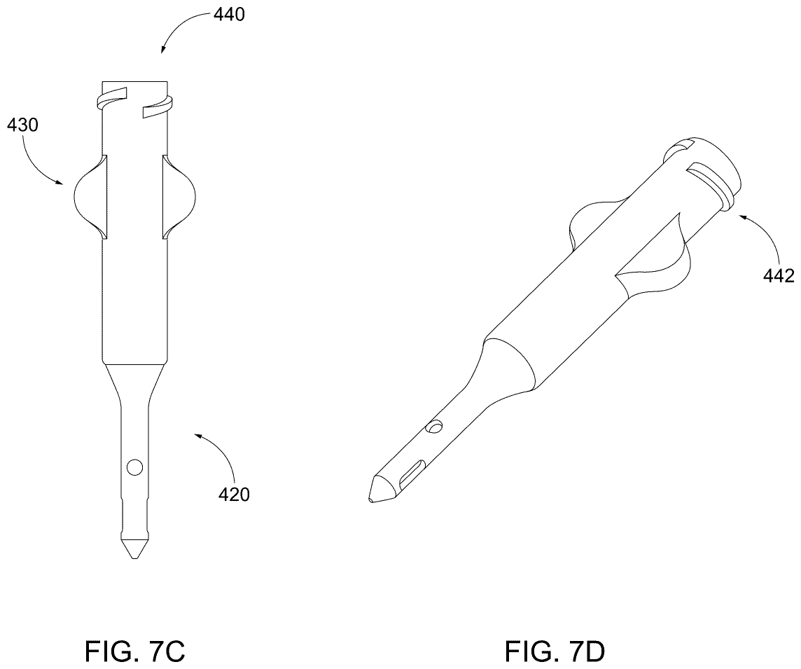

It is contemplated that a system for a spinal surgical procedure can include pedicle tips for introducing a medical product in paste form to a hole formed in bone. For example, a pedicle tip delivers a hemostatic paste to a hole formed for a pedicle screw, or other anchor, in a vertebra or another bone during surgery. One or more pedicle tips or nozzles can be provided. The pedicle tips can be provided in various configurations. One example of a pedicle tip is shown in FIGS. 7A-7D. The pedicle tip 400 has at least one orifice 410, and in certain embodiments up to four orifices, for delivering the paste material.

In certain embodiments, the pedicle tip 400 has a tapered elongate portion 420 for ease of insertion and sealing to avoid the medical product/paste material from oozing out of the hole (extravasation). In certain embodiments, the pedicle tip 400 has features 430 for ease of grasping by hand or by an instrument. In certain embodiments, the pedicle tip 400 has an open upper end 440 that receives the medical product. The upper end 440 can have a connector 442 for attaching a syringe containing the medical product.

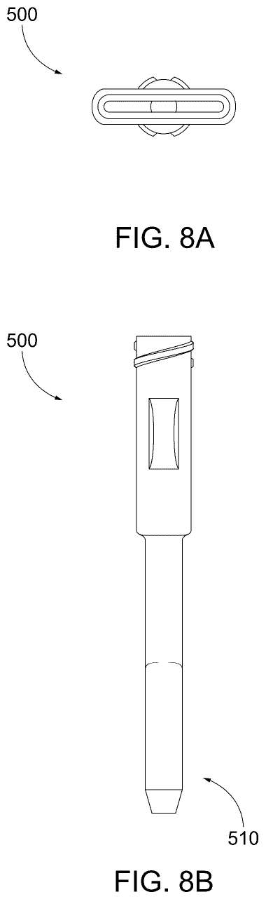

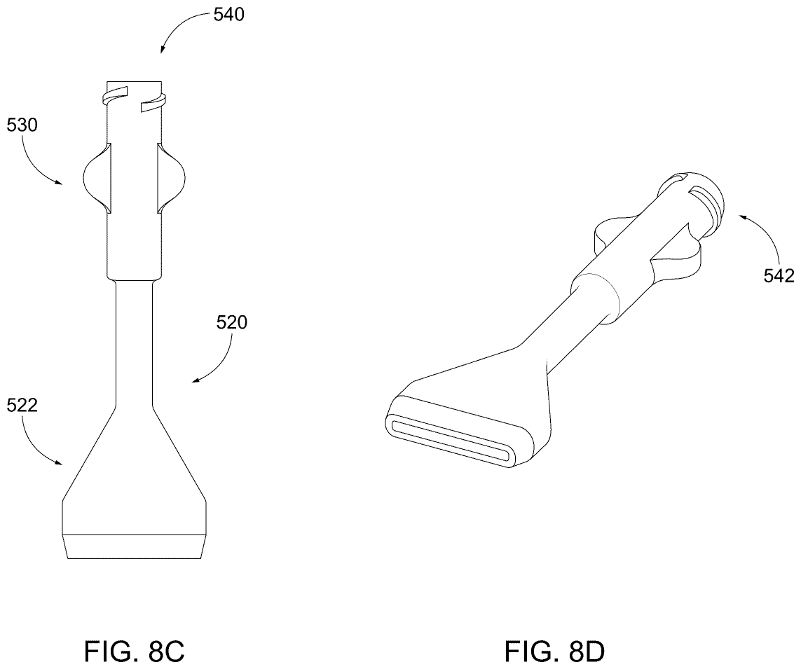

A further example of a configuration for a pedicle tip is shown in FIGS. 8A-8D. The pedicle tip 500 has at least one orifice 510 for delivering the paste material. In certain embodiments, the pedicle tip 500 has a tapered elongate portion 520 for ease of insertion and sealing to avoid the medical product/paste material from extravasating out of the hole. The pedicle tip 500 has a distal end 522 with a wide, flat shape. In certain embodiments, the pedicle tip 500 has features 530 for ease of grasping by hand or by an instrument. In certain embodiments, the pedicle tip 500 has an open upper end 540 that receives the medical product. The upper end 540 can have a connector 542 for attaching a syringe containing the medical product.

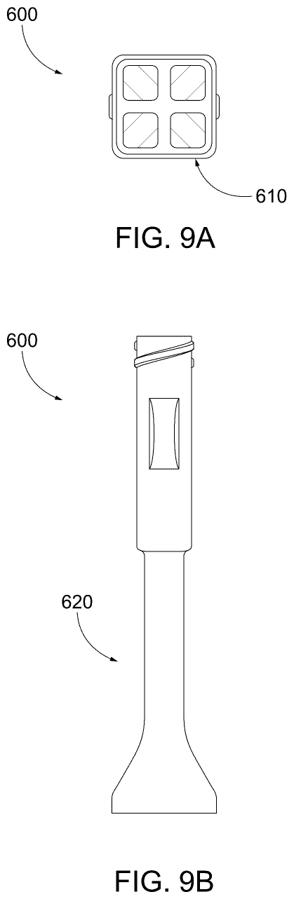

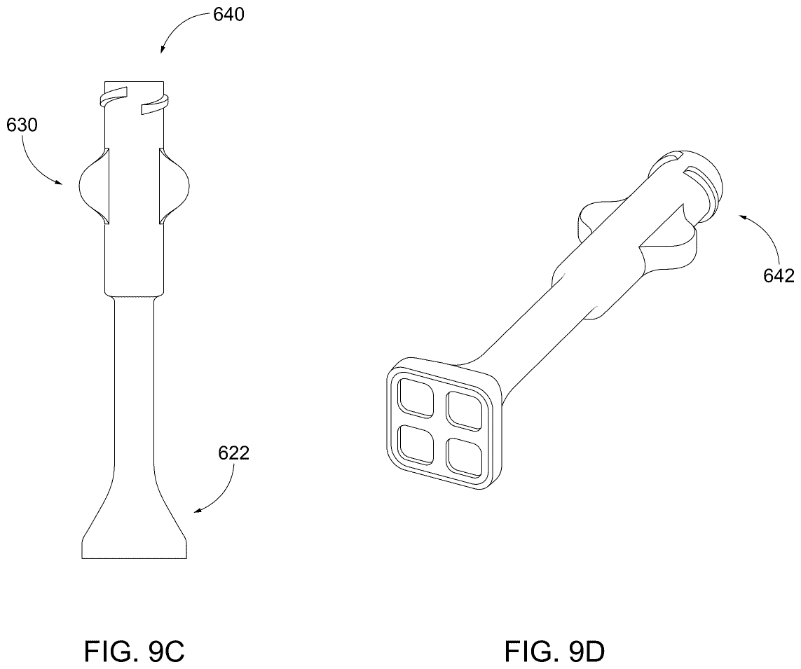

A further example of a configuration for a pedicle tip is shown in FIGS. 9A-D. The pedicle tip 600 has at least one orifice 610 (and in certain embodiments, four orifices) for delivering the paste material. In certain embodiments, the pedicle tip 600 has a tapered elongate portion 620 for ease of insertion and sealing to avoid the medical product/paste material from extravasating out of the hole. The pedicle tip 600 has a distal end 622 with a square or rectangular, flat shape. In certain embodiments, the pedicle tip 600 has features 630 for ease of grasping by hand or by an instrument. In certain embodiments, the pedicle tip 600 has an open upper end 640 that receives the medical product. The upper end 640 can have a connector 642 for attaching a syringe containing the medical product.

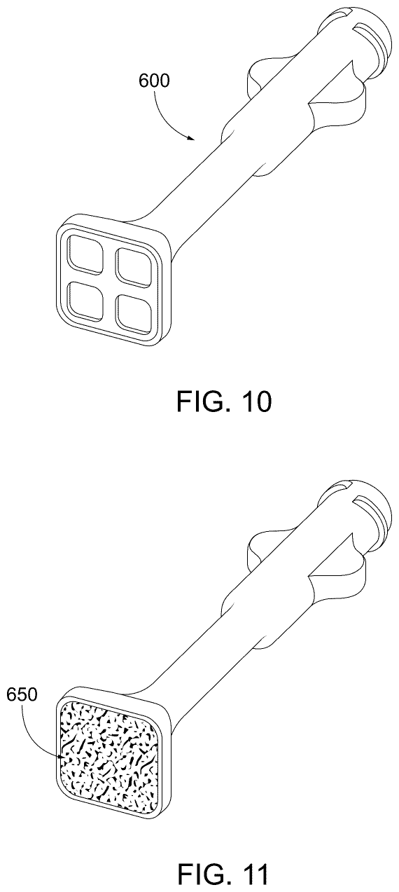

FIGS. 10 and 11 are additional views of the pedicle tip 600 of FIGS. 9A-9D, FIG. 9D showing the pedicle tip with a sponge 650 attached.

It is contemplated that a system for spinal surgery can include a kit having a medical mixer apparatus and one or more pedicle tips. The kit may include a medical product in a vial, such as, for example, a vial of thrombin. The kit may include a vial or syringe of saline. The kit may further include a syringe of gelatin. A paste of thrombin can be prepared, as discussed above, using the medical mixer apparatus. The paste can be disposed in a syringe. The one or more pedicle tips can have a connector for attaching the syringe thereto. The pedicle tips can include a tapered tip, like tip 400, a wide flat tip, like tip 500, and/or a square tip, like tip 600. Other shapes and configurations are contemplated.

The medical mixer apparatus can have, in certain embodiments, one or more of the following features and benefits. The mixer can facilitate use of non-sterile materials such as thrombin and other hemostatic agents (such as Tranexamic Acid) that are packaged in non-sterile containers by creating a sterile environment for hemostatic agent integration into sterile gelatin and sterile saline to create a sterile hemostatic agent for use in the sterile field in the operating room and for application into wounds, incisions, and lacerations. The mixer can facilitate combining bone products, biologic agents, hemostatic agents, and other materials to create end products that allow for bone fusion, bone union and hemostasis.

The mixer can allow for rapid hydration of demineralized bone matrix powders and demineralized bone matrix fibers, integration of antibiotics into demineralized bone matrix products and hemostatic agents, and/or integration of growth factors, analgesics, or other drugs requiring targeted delivery to the surgical field.

In embodiments, the mixer has baffles, or other agitation features to aid in creating turbulence to minimize mixing time and improve passages of gelatin.

The mixer can allow selective amount of hydration to adjust viscosity of gelatin/thrombin mix. The mixer can be a closed system that prevents product loss of thrombin-saline fluid and loss of gelatin.

In embodiments, the mixer is a closed system that integrates multiple steps needed to create existing hemostatic products, thereby 1) reducing the steps needed to create the hemostatic product, 2) reducing the time needed to create the hemostatic product, 3) reducing waste of the hemostatic product components, 4) reducing user confusion in following the steps need to create the hemostatic product, 5) and creating a hemostatic product that has more consistent viscosity, more consistent handling characteristics, and more consistent hemostatic product efficacy.

The mixer may allow for more consistent viscosity of hemostatic agent because product component loss is minimized, thereby improving the handling and performance profile of the hemostatic product.

In embodiments, the mixer is arranged to reduce the amount of air trapped in the apparatus thereby reducing product sputtering during application of hemostatic product, and reducing hemostatic product waste during hemostatic product application.

The mixer knob may allow mechanical advantage which facilitates perforating the septum of the vial. In embodiments, the pointed spike in the vial chamber is designed to avoid coring of the septum. The vial chamber may be arranged to ensure the vial is vertically aligned with the septum spike to ensure proper perforation.

In embodiments, a system includes a pedicle tip; the pedicle tip can allow sealing of the hole in the pedicle to ensure all the material (such as Hemostatic Paste (“HP”)) stays in the pedicle, thereby reducing hemostatic product waste and improving hemostatic product efficacy. The pedicle tip can have delivery perforations in multiple quadrants (such as, for example, in 4 quadrants) to ensure even distribution of hemostatic product and reducing hemostatic product extravasation.

In an example, a pedicle tip is tapered to facilitate introduction into the pedicle hole.

In another example, a square tip allows hemostatic product to be integrated into irregular bony surfaces, thereby reducing hemostatic product waste and improving hemostatic product efficacy; such a tip can include a sponge.

In an example, a wide tip allows hemostatic product to be placed along a wide surface of bone or soft tissue, thereby reducing hemostatic product waste and improving hemostatic product efficacy.

In another example, a square tip allows a square of HP to be deposited in a similar manner as a cottonoid, thereby reducing hemostatic product waste and improving hemostatic product efficacy. In an example, the square shaped tip can be a 10 mm×10 mm square.

Although various embodiments of the invention have been disclosed herein, the invention is not limited to only these embodiments. It is readily recognized that various modifications and alterations of these embodiments will be apparent to those skilled in the art. However, it is expressly noted that such modifications and alterations are within the scope and spirit of the present disclosure. The foregoing discussion of the disclosure has been presented for purposes of illustration and description.

What is claimed is:

1. A medical mixer apparatus, comprising:
a vial holder having a chamber;
a first connector; and
an adapter defining a first passageway, the adapter being movable to a position in which the first passageway extends from the first connector to the chamber.

2. The apparatus according to claim 1, further comprising a vial wherein the vial can be disposed in the vial holder.

3. The apparatus according to claim 2, further comprising a cap attachable to a first end of the vial holder.

4. The apparatus according to claim 3, wherein the vial holder has a second end opposite the first end and a spike at the second end.

5. The apparatus according to claim 1, wherein the adapter has a second passageway and the apparatus further comprises a second connector.

6. The apparatus according to claim 5, wherein the adapter is movable from a first position in which the chamber and the first connector are aligned with the first passageway to a second position in which the first connector and second connector are aligned with the second passageway.

7. The apparatus according to claim 6, wherein at least one of the first connector and second connector is arranged to connect to a syringe.

8. The apparatus according to claim 6, wherein the first connector is arranged to connect to a first syringe and the second connector is arranged to connect to a second syringe.

9. The apparatus according to claim 7, wherein the syringe contains saline.

10. The apparatus according to claim 6, wherein the second passageway has at least one bump defined therein.

11. The apparatus according to claim 6, wherein the second passageway has at least one curved ridge defined therein.

12. The apparatus according to claim 1, wherein the adapter is rotatable.

13. The apparatus according to claim 6, wherein the second passageway is disposed beneath the first passageway.

14. The apparatus according to claim 13, wherein the adapter is rotatable and the adapter moves up with respect to the vial holder when rotated to align the first connector and second connector with the second passageway.

15. A kit, comprising saline in a first syringe, another substance in a vial, and the medical mixer apparatus according to claim 1.

16. A method of mixing a medical product, comprising providing the kit according to claim 15, and mixing the saline and the other substance using the medical mixer apparatus.

17. The method according to claim 16, further comprising rotating the adapter to move the adapter.

18. A kit, comprising saline in a first syringe, another substance in a vial, a second syringe, and the medical mixer apparatus according to claim 16.

19. A method of mixing a medical product, comprising providing the kit according to claim 18, and mixing the saline and the other substance using the medical mixer apparatus.

20. The method according to claim 19, further comprising rotating the adapter to move the adapter from the first position to the second position.
